(19) United States
(12) Patent Application Publication
(10) Pub. No.: US 2010/0003201 A1
(43) Pub. Date: Jan. 7, 2010

(54) Silicone composition

(75) Inventor: Roy Uwe Wahl, Teaneck, NJ (US)

Correspondence Address: MOMENTIVE PERFORMANCE MATERIALS INC.;c/o Dilworth & Barrese, LLP, 1000 Woodbury Road, Suite 405, Woodbury, NY, 11797, US

(73) Assignee: Momentive Performance Materials, Inc.

(21) Appl. No.: 12/217,548

(22) Filed: Jul. 7, 2008

(51) Int. Cl. A61K 8/30; A61K 31/695; A61Q 19/00; A61Q 17/00; A61Q 15/00; A61Q 11/00; A61Q 5/00; A61Q 3/00; A61Q 1/00

(52) U.S. Cl. 424/49; 514/63; 424/59; 424/62; 424/63; 424/64; 424/65; 424/69; 424/70.1; 424/61

(57) ABSTRACT

This invention relates to silicone-containing compositions, method of making said silicone-containing compositions, and to personal care and other products based on such compositions.

### BACKGROUND OF INVENTION

This invention relates to silicone-containing compositions, a process for making said silicone-containing compositions, and to personal care products based on such compositions.

When formulating personal care products such as cosmetics problems may arise due to these types of materials being difficult to disperse resulting in lumping of the carbomer, or sticky gobs and otherwise unreliable carbomer dispersion resulting in unpredictable and non-reproductive viscosities in the final product formation. In addition the time it takes to swell the carbomer can be adversely affected by inadequate dispersion of this material. Meticulous dispersion techniques are thus necessary in order to disperse most carbomer and often long wetting times, e.g., greater than 20 minutes and up to 5 hours may be necessary to achieve an acceptable level dispersion.

It has therefore been desirable to provide a carbomer-containing composition exhibiting a high level of dispersion of its carbomer component, which is efficiently and effectively achieved employing conventional equipment and standard manufacturing practices.

### SUMMARY OF THE INVENTION

In accordance with the present invention, a polysiloxane-based composition is provided which comprises:
a) at least one polysiloxane of the general formula (1):

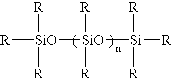

wherein each R is the same or different straight or branched open chain alkyl group containing from 1 to 12 carbon atoms, n is from 0 to 5;
b) at least one carbomer; and
c) water or aqueous solution comprising at least 1 weight percent of the total weight of (a), (b) and (c).

The present invention further provides a process for producing a polysiloxane-based composition comprising:

i) mixing:
a) at least one polysiloxane of the general formula (1):

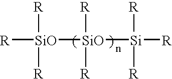

wherein each R is the same or different straight or branched open chain alkyl group containing from 1 to 12 carbon atoms, n is 0 to 5;
b) at least one carbomer; and,
c) water or an aqueous solution comprising at least 1 weight percent of the total weight of (a), (b) and (c).

The present invention provides a carbomer-containing composition exhibiting a high level of dispersion of its carbomer component, useful in the preparation of various personal care products, cosmetics and the like. The inventive composition and formulations made therefrom are efficiently and effectively realized employing conventional equipment and standard manufacturing practices.

### DETAILED DESCRIPTION OF THE INVENTION

The singular forms “a,” “an” and “the” include plural referents unless the context clearly dictates otherwise. The endpoints of all ranges reciting the same characteristic are independently combinable and inclusive of the recited endpoint. All references are incorporated herein by reference.

The modifier “about” used in connection with a quantity is inclusive of the stated value and has the meaning dictated by the context (e.g., includes the tolerance ranges associated with measurement of the particular quantity).

“Optional” or “optionally” means that the subsequently described event or circumstance may or may not occur, or that the subsequently identified material may or may not be present, and that the description includes instances where the event or circumstance occurs or where the material is present, and instances where the event or circumstance does not occur or the material is not present.

In each generic structural chemical formula described and/or claimed herein wherein two or more substituents (inclusive of such terms as “groups,” “functional groups,” “radicals” and “moieties”) are each defined as any one of several specified members, the structural formula shall be regarded as including all possible combinations of members defining all such substituents (subgenuses) and as disclosing each combination (subgenus) as if it were individually set forth.

It will be understood herein that all uses of the term centistokes were measured at 25 degrees celsius.

It will be understood herein that all specific, more specific and most specific ranges recited herein comprise all sub-ranges therebetween.

A desired viscosity as used herein can vary greatly depending upon application of polysiloxane-based composition described herein. According to an embodiment of the invention, the silicone fluid, i.e., polysiloxane (a) has a viscosity of less than 5000 cSt. In one specific embodiment, polysiloxane-based composition can be used in personal care formulations as described herein. A personal care formulation can also have any viscosity that would be desirable for the particular personal care formulation.

The silicone fluid, i.e., polysiloxane (a) of the polysiloxane-based composition, can be selected from among any of the known and conventional silicone fluids heretofore employed in personal care products. For example, the silicone fluid that can be an organopolysiloxane, a silicone copolyol, a disiloxane, trisiloxane, tetrasiloxane, or a trimethicone, an alkylsiloxane or a cyclopolysiloxane, or combinations thereof.

Representative silicone fluids include branched, unbranched, linear or cyclic silicone fluids such as those having a viscosity≦8 centistokes, and having, for example from 2 to 7 silicon atoms, these silicones optionally comprising alkyl, polyether- or alkoxy groups having from 1 to 12 carbon atoms. Some non-limiting examples of silicone fluids which can be used in the invention include octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane, capryl methicone, PEG/PPG 5/3 Methicone, and mixtures thereof.

Also useful herein are silicone fluids such as, for example, polydimethylsiloxanes (PDMS), polydimethylsiloxanes comprising alkyl, polyether- or alkoxy groups, pendant and/or at the silicone chain end, the alkyl and alkoxy groups each having from 1 to 12 carbon atoms, phenylated silicones such as ethylmethicone, heptylmethicone, hexylmethicone, propylmethicone, isopropylmethicone, heptylmethicone, sec-butylmethicone, tert-butylmethicone, pentylmethicone, phenyltrimethicones, phenyldimethicones, phenyltrimethylsiloxydiphenylsiloxanes, diphenyldimethicones, diphenylmethyl-diphenyltrisiloxanes and (2-phenylethyl)trimethylsiloxy-silicates.

Although disiloxanes or tetrasiloxanes can be used for polysiloxane (a), according to one specific embodiment, polysiloxane (a) is a trisiloxane. Some non-limiting examples of trisiloxane of general formula (1) are linear alkyltrisiloxanes selected from the group consisting of ethyltrisiloxane, such as the non-limiting examples of 1,1,1,3,5,5,5-heptamethyl-3-ethyltrisiloxane; octyltrisiloxane, such as the non-limiting example of 1,1,1,3,5,5,5-heptamethyl-3-octyltrisiloxane; hexyltrisiloxane such as the non-limiting example of 1,1,1,3,5,5,5-heptamethyl-3-hexyltrisiloxane, and combinations thereof. In one specific embodiment herein trisiloxane of general formula (1) can be at least one trisiloxane such as those described in U.S. Patent Application Publication No. 2004/0197284A1 which is incorporated by reference herein in its entirety. In another specific embodiment herein trisiloxane of the general formula (1) can be at least one trisiloxane such as those described in U.S. Patent Application Publication No. 2005/0069564A1 the contents of which are incorporated by reference herein in its entirety. In yet one even more specific embodiment, trisiloxane of the general formula (1) can be selected from the group consisting of 3-pentyl-1,1,1,3,5,5,5-heptamethyltrisiloxane; 1,1,1,3,5,5,5-heptamethyl-3-octyltrisiloxane, sold, for example, under the name “Silsoft 034“by Momentive Performance Materials Inc.; 1,1,1,3,5,5,5-heptamethyl-3-hexyltrisiloxane; 3-(1-ethylbutyl)-1,1,1,3,5,5,5-heptamethyltrisiloxane; 1,1,1,3,5,5,5-heptamethyl-3-(1-methylpentyl)trisiloxane; 1,1,1,3,5,5,5-heptamethyl-3-(1-methylpropyl)trisiloxane; 3-(1,1-dimethylethyl)-1,1,1,3,5,5,5-heptamethyltrisiloxane; 1,1,1,5,5,5-hexamethyl-3,3-bis(1-methylethyl)trisiloxane; 3-(3,3-dimethylbutyl)-1,1,1,3,5,5,5-heptamethyltrisiloxane; 1,1,1,3,5,5,5-heptamethyl-3-(3-methylbutyl)trisiloxane; 1,1,1,3,5,5,5-heptamethyl-3-(3-methylpentyl)trisiloxane; 1,1,1,3,5,5,5-heptamethyl-3-(2-methylpropyl)trisiloxane; 1,1,1,3,5,5,5-heptamethyl-3-propyltrisiloxane; 3-isohexyl-1,1,1,3,5,5,5-heptamethyltrisiloxane; 3-tert-pentyl-1,1,1,3,5,5,5-heptamethyltrisiloxane; 3-neo-pentyl-1,1,1,3,5,5,5-heptamethyltrisiloxane; 1,1,1,5,5,5-hexamethyl-3,3-dipropyltrisiloxane; 3,3-diethyl-1,1,1,5,5,5-hexamethyltrisiloxane; 3,3-dibutyl-1,1,1,5,5,5-hexamethyltrisiloxane; 3-ethyl-1,1,1,3,5,5,5-heptamethyltrisiloxane; 3-heptyl-1,1,1,3,5,5,5-heptamethyltrisiloxane; 1,1,1,5,5,5-hexamethyl-3,3-diethyltrisiloxane; 1,1,1,3,5,5,5-heptamethyl-3-butyltrisiloxane and combinations thereof.

According to one specific embodiment of the invention, polysiloxane (a) of the polysiloxane-based composition has the following formula (2):

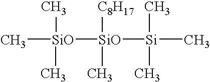

According to another specific embodiment of the invention, polysiloxane (a) of the polysiloxane-based composition has the following formula (3):

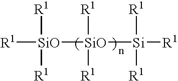

wherein R¹ is the same or different alkyl of 1 to 12 carbon atoms, n is 0 to 5, and according to another embodiment of the invention R¹ is the same or different alkyl having from 6 to 10 carbon atoms, and n is 0 to 5.

According to yet another embodiment of the invention, polysiloxane (a) of the polysiloxane-based composition has the following formula (4):

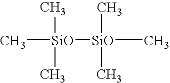

Additionally, polysiloxane (a) can be any one of a number of commercially available materials such as, but not limited to, Silsoft 034 from Momentive Performance Materials Inc., Toray FZ-3196 from Dow Corning Inc or SilCare Silicone 41M15 from Clariant Inc, Sibrid AM 108 from Gelest, or combinations thereof Also, mixtures such as, but not limited to, Hydrobrite 2000 Gel (from Chemtura formerly Witco) or SilCare 51M15 Trimethylsiloxysilicate in Caprylylmethicone (from Clariant) are included as polysiloxanes in the sense of the invention.

The carbomers, i.e., component (b), of the polysiloxane-based composition of the present invention include synthetic high molecular weight polymers of acrylic acid cross linked with either allyl esters of sucrose, pentaerythritol, or propylene. Examples of carbomers include, for example, hydroxypropylethylene diamine carbomer and triethanolamine cabomer. Salts of the carbomer may be complete salts (where all of the acid groups have been neutralized) or partial salts (in which only a proportion of the acid groups have been neutralized). Salts of the carbomer will be understood to include complete salts, partial salts or mixtures thereof. Preferred, but not limited to, are salts of carbomer that are mono or divalent salts, most preferably sodium, calcium or potassium salts. Commercially available non-limiting examples of such carbomers are those sold under tradenames such as Carbopol from Noveon (formerly BF Goodrich), Acritamer (RITA), Julon (Nihon Junyaku), Polacril (Biophil), Synthalen (3V Inc), Tego (Evonik, formerly Degussa Goldschmidt) as well as mixtures thereof. Carbomers can be used as structurants or thickeners in personal care products.

Structurants or thickener of the invention can be of carbomer nature, or of non-carbomer nature, and include, but are not limited to, any form of acrylates copolymers, gums including guar gums and xanthan gums, cellulose derivatives, starches, aluminium silica clays such as kaolin and other clays. Other aqueous thickeners or structurants include those defined and listed in the International Cosmetic Ingredient Dictionary and Handbook, a publication well known to artisans in the cosmetic field. These would include, but not be limited to, materials available in the marketplace under trade names such as Carbopol or Pemulen by Noveon Inc., Jaguar by Rhodia Inc., Veegum by RT Vanderbilt Inc., Bentonite by A & E Connock Inc. or by Whittaker Clark & Daniels Inc., Bentone Gel by Elementis Specialties Inc., Polargel by American Colloid Consumer Specialties Inc., and Keltrol by CP Kelco Inc., and Natrosol by Hercules Inc.

Component (c) of the inventive composition can be water or an aqueous solution. As contemplated herein, an aqueous solution is a solution in which the solvent is water, e.g., brine. Other non-limiting examples of component (c) include aqueous dispersions, such as, for example, that of a pigment in water, or slurries, or any isotropic- or non-isotropic system that has water as the main component.

Ranges of components (a), (b) and (c), of the inventive composition in weight percent of the total composition are as follows:

| Component | Range of Amount |
|---|---|
| polysiloxane (component (a)) | 0.01-99 |
| | 0.1-15 |
| | 1-5 |
| carbomer (component (b)) | 0.0001-40 |
| | 0.001-10 |
| | 0.02-5 |
| water or aqueous solution (component (c)) | 1-99.5 |
| | 20-99 |
| | 60-98 |

Additional components can be added to the polysiloxane-based composition of the invention in order to prepare, for example, personal care products, homecare products, other consumer products, medical products and the like. in known and conventional amounts. Examples of the additional components include surfactants, emulsifiers, solvents, emollients, moisturizers, humectants, pigments, colorants, fragrances, biocides, preservatives, chelating agents, antioxidants, anti-microbial agents, anti-fungal agents, antiperspirant agents, exfoliants, hormones, enzymes, medicinal compounds, vitamins, alpha-hydroxy acids, beta-hydroxy acids, retinols, niacinamide, skin lightening agents, salts, electrolytes, alcohols, polyols, absorbing agents for ultraviolet radiation, botanical extracts, organic oils, waxes, film formers, thickening agents, particulate fillers, silicones, clays, plasticizers, humectants, occlusives, sensory enhancers, esters, other resins and film formers or film forming emulsifiers or high refractive index materials, and the like, and combinations comprising at least one of the foregoing components. The addition components can also include bleaches, insecticides, demulsifiers and many other ingredients of various functionality depending on the targeted industrial- or end user application.

Examples of personal care products in which the present invention can be used to prepare include such products as deodorants, antiperspirants, antiperspirant/deodorants, including sprays, sticks and roll-on products, shaving products, skin lotions, moisturizers, toners, bath products, cleansing products, shampoos, conditioners, combined shampoo/conditioners, mousses, styling gels, hair sprays, hair dyes, hair color products, hair bleaches, waving products, hair straighteners, nail polish, nail polish remover, nail creams and lotions, cuticle softeners, sunscreen, insect repellent, anti-aging products, lipsticks, foundations, face powders, eye liners, eye shadows, blushes, makeup, mascaras, moisturizing preparations, foundations, body and hand preparations, skin care preparations, face and neck preparations, tonics, dressings, hair grooming aids, aerosol fixatives, fragrance preparations, aftershaves, make-up preparations, soft focus applications, night and day skin care preparations, non-coloring hair preparations, tanning preparations, synthetic and non-synthetic soap bars, hand liquids, nose strips, non-woven applications for personal care, baby lotions, baby baths and shampoos, baby conditioners, shaving preparations, cucumber slices, skin pads, make-up removers, facial cleansing products, cold creams, sunscreen products, mousses, spritzes, paste masks and muds, face masks, colognes and toilet waters, hair cuticle coats, shower gels, face and body washes, personal care rinse-off products, gels, foam baths, scrubbing cleansers, astringents, nail conditioners, eye shadow sticks, powders for face or eye, lip balms, lip glosses, hair care pump sprays and other non-aerosol sprays, hair-frizz-control gels, hair leave-in conditioners, hair pomades, hair de-tangling products, hair fixatives, hair bleach products, skin lotions, pre-shaves and pre-electric shaves, anhydrous creams and lotions, oil/water, water/oil, multiple and macro and micro emulsions, water-resistant creams and lotions, anti-acne preparations, mouth-washes, massage oils, toothpastes, clear gels and sticks, ointment bases, topical wound-healing products, aerosol talcs, barrier sprays, vitamin and anti-aging preparations, herbal-extract preparations, bath salts, bath and body milks, hair styling aids, hair-, eye-, nail-and skin-soft solid applications, controlled-release personal care products, hair conditioning mists, skin care moisturizing mists, skin wipes, pore skin wipes, pore cleaners, blemish reducers, skin exfoliators, skin desquamation enhancers, skin towelettes and cloths, depilatory preparations, personal care lubricants, nail coloring preparations, drug delivery systems for topical application of medicinal compositions that are to be applied to the skin and combinations comprising at least one of the foregoing applications.

### EXAMPLES

It is important to mention that, even though a batch reactor setup of a two liter reactor volume with a u-shaped universal anchor mixer was used (see experiments, herein below), the nature of this invention is independent of the processing equipment or processing conditions employed, since the carbomer dispersing or reactor wetting properties of caprylyl methicone, for example, are intrinsic to its molecular structure.

It is furthermore important to mention that the nature of this invention is independent of the order of addition of the water, caprylyl methicone or carbomer, even though charging the kettle with caprylyl methicone first is a preferred way of operating.

Preparation of Example 1 (as representative of the inventive polysiloxane-based composition, the weight percent of each component is presented in Table 1) containing polysiloxane, carbomer, and water was accomplished by adding 24.00 g of caprylyl methicone into to a 2 liter IKA Miniplant 2 reactor at 25° C., followed by addition of 3.60 g of carbomer, Carbopol 934 (manufactured by Noveon, formerly BF Goodrich) (i.e., carbopol 934, is prepared from acrylic acid cross linked with poly allyl sucrose), and 701.28 g of deionized water and mixing at 61 rpm.

TABLE 1
All percents being weight percent.
Example 1

| Component (a) Caprylyl | Component (b) | Component (c) |
|---|---|---|
| Methicone [%] | Carbomer [%] | Water [%] |
| 3.29 | 0.49 | 96.21 |

Example 1 was used to prepare the following the finished suncare formulation presented in Table 2, below.

TABLE 2

| | | Actual charges for |
|---|---|---|
| Ingredients | [wt %] | 1200 g batch: [g] |
| Isopropyl Myristate | 4.00 | 48.00 |
| Caprylic Capric Triglycerides | 4.00 | 48.00 |
| Ethylhexyl Methoxycinnamate | 7.50 | 90.00 |
| Ethylhexyl Salicylate | 5.00 | 60.00 |
| Sorbitan Laurate | 3.50 | 42.00 |
| Cetearyl Alcohol | 2.00 | 24.00 |
| Ceteareth-20 | 2.00 | 24.00 |
| Butylmethoxydibenzoylmethane | 1.00 | 12.00 |
| Cyclopentasiloxane (and) C30-45 | 5.00 | 60.00 |
| Alkyl Cetearyl Dimethicone | | |
| Crosspolymer | | |
| Caprylyl Methicone | 2.00 | 24.00 |
| Cyclopentasiloxane (and) | 2.00 | 24.00 |
| Trimethylsiloxysilicate | | |
| Polymethylsilsesquioxane | 2.20 | 26.40 |
| Inulin Lauryl Carbamate | 0.50 | 6.00 |
| DI Water | 58.52 | 701.28 |
| Tetrasodium EDTA | 0.08 | 0.96 |
| Carbomer (Carbopol 934) | 0.30 | 3.60 |
| Triethanolamine | 0.34 | 4.07 |
| DMDM Hydantoin (and) | 0.06 | 0.75 |
| Iodopropynyl Butylcarbamate (and) | | |
| Butylene Glycol (and) Water | | |
| Triethanolamine | 0.08 | 0.94 |

The suncare formulation was prepared as follows: To inventive composition Example 1, at 70 rpm mixing speed and after 1 hour at 60° C. the tetrasodium EDTA is added. Then caprylic/capric triglycerides, ethylhexyl methoxycinnamate, ethylhexyl salicylate and the isopropyl myristate are added. Then the butylmethoxydibenzoylmethane is added. Then triethanolamine is added until pH goes from 4.10 to 6.31. After 15 min, Cyclopentasiloxane (and) C30-45 Alkyl Cetearyl Dimethicone Crosspolymer and Cyclopentasiloxane (and) Trimethylsiloxysilicate are added. Then, the polymethylsilsesquioxane is added. Then the rpm is increased to 110 rpm. Then the cetearyl alcohol is added. Then the sorbitan laurate is added. Then the inulin lauryl carbamate is added. Then the rpm is increased to 130 rpm. The temp is increased to 65 C. After 30 min, the system is allowed to cool while stirring. Then the DMDM hydantoin (and) iodopropynyl butylcarbamate (and) butylene glycol (and) water is added, and the pH is brought to 6.8 with a second addition of triethanolamine.

The use of caprylyl methicone as component (a) of the inventive composition provided a finished suncare formulation that was very smooth, homogeneous and stable having a viscosity of 7500-8000 cps, measured at 25° C. at 10 rpm with a TE-spindle using a Brookfield RV DII viscometer with a helipath.

Addition of caprylyl methicone directly to the suncare formulation provided a much lower viscosity of the formulation due to the lumping of the carbomer in the reactor.

According to a specific embodiment of the invention, the carbomer is mixed with caprylyl methicone prior to its mixing with water, which provided carbomer finely dispersed in caprylyl methicone, with an average particle size of less than 100 microns, and a median size of around 20 to 50 microns. However, dispersing the carbomer in water before adding it to the caprylyl methicone, provided an average particle size of around 0.2 to 2.5 centimeters, with a median size of around 1 to 1.5 centimeters.

While the invention has been described with reference to a preferred embodiment, those skilled in the art will understand that various changes may be made and equivalents may be substituted for elements thereof without departing from the scope of the invention. It is intended that the invention not be limited to the particular embodiment disclosed as the best mode for carrying out this invention, but that the invention will include all embodiments falling within the scope of the appended claims. All citations referred herein are expressly incorporated herein by reference.

1. A polysiloxane-based composition which comprises:
a) at least one polysiloxane of the general formula: 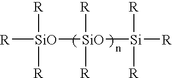
wherein each R is the same or different straight or branched open chain alkyl group containing from 1 to 12 carbon atoms, n is 0 to 5;
b) at least one carbomer; and
c) water or an aqueous solution comprising at least 1 weight percent of the total weight of (a), (b) and (c).

2. The polysiloxane-based composition of claim 1 wherein polysiloxane (a) is a polysiloxane having the formula: 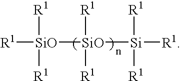
wherein R¹ is the same or different alkyl group containing from 6 to 10 carbon atoms, and n is 0 to 5.

3. The polysiloxane-based composition of claim 1 wherein polysiloxane (a) is a polysiloxane having the formula:

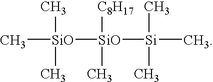

4. The polysiloxane-based composition of claim 1 wherein polysiloxane (a) is a polysiloxane having the formula:

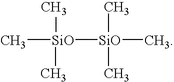

5. The polysiloxane-based composition of claim 1 wherein polysiloxane (a) is present in an amount that ranges 0.01 to 99 weight percent of the total composition, carbomer (b) is present in an amount that ranges from 0.0001 to 40 weight percent of the total composition, and water or aqueous solution 1 to 99.5 weight percent of the total composition.

6. The polysiloxane-based composition of claim 1 wherein polysiloxane (a) is present in an amount that ranges 0.1 to 15 weight percent of the total composition, carbomer (b) is present in an amount that ranges from 0.001 to 10 weight percent of the total composition, and water or aqueous solution 20 to 99 weight percent of the total composition.

7. The polysiloxane-based composition of claim 1 wherein polysiloxane (a) is present in an amount that ranges 1 to 5 weight percent of the total composition, carbomer (b) is present in an amount that ranges from 0.02 to 5 weight percent of the total composition, and water or aqueous solution 60 to 98 weight percent of the total composition.

8. The polysiloxane-based composition of claim 1 wherein polysiloxane (a) is at least one selected from the group consisting of 1,1,1,3,5,5,5-heptamethyl-3-ethyltrisiloxane; 1,1,1,3,5,5,5-heptamethyl-3-octyltrisiloxane; 1,1,1,3,5,5,5-heptamethyl-3-hexyltrisiloxane, 3-pentyl-1,1,1,3,5,5,5-heptamethyltrisiloxane; 1,1,1,3,5,5,5-heptamethyl-3-octyltrisiloxane, 1,1,1,3,5,5,5-heptamethyl-3-hexyltrisiloxane; 3-(1-ethylbutyl)-1,1,1,3,5,5,5-heptamethyltrisiloxane; 1,1,1,3,5,5,5-heptamethyl-3-(1-methylpentyl)trisiloxane; 1,1,1,3,5,5,5-heptamethyl-3-(1-methylpropyl)trisiloxane; 3-(1,1-dimethylethyl)-1,1,1,3,5,5,5-heptamethyltrisiloxane; 1,1,1,5,5,5-hexamethyl-3,3-bis(1-methylethyl)trisiloxane; 3-(3,3-dimethylbutyl)-1,1,1,3,5,5,5-heptamethyltrisiloxane; 1,1,1,3,5,5,5-heptamethyl-3-(3-methylbutyl)trisiloxane; 1,1,1,3,5,5,5-heptamethyl-3-(3-methylpentyl)trisiloxane; 1,1,1,3,5,5,5-heptamethyl-3-(2-methylpropyl)trisiloxane; 1,1,1,3,5,5,5-heptamethyl-3-propyltrisiloxane; 3-isohexyl-1,1,1,3,5,5,5-heptamethyltrisiloxane; 3-tert-pentyl-1,1,1,3,5,5,5-heptamethyltrisiloxane; 3-neo-pentyl-1,1,1,3,5,5,5-heptamethyltrisiloxane; 1,1,1,5,5,5-hexamethyl-3,3-dipropyltrisiloxane; 3,3-diethyl-1,1,1,5,5,5-hexamethyltrisiloxane; 3,3-dibutyl-1,1,1,5,5,5-hexamethyltrisiloxane; 3-ethyl-1,1,1,3,5,5,5-heptamethyltrisiloxane; 3-heptyl-1,1,1,3,5,5,5-heptamethyltrisiloxane; 1,1,1,5,5,5-hexamethyl-3,3-diethyltrisiloxane; 1,1,1,3,5,5,5-heptamethyl-3-butyltrisiloxane, and PEG/PPG 5/3 Methicone.

9. The polysiloxane-based composition of claim 1 wherein the carbomer (b) is at least one synthetic high molecular weight polymer of acrylic acid cross linked with allyl esters of sucrose, pentaerythritol, or propylene.

10. The polysiloxane-based composition of claim 1 wherein carbomer (b) is a non-carbomer structurant or thickener.

11. The polysiloxane-based composition of claim 10 wherein non-carbomer (b) is at least one selected from the group consisting of gums, cellulose derivatives, starches, and clays.

12. A personal care formulation comprising the polysiloxane-based composition of claim 1 where the personal care formulation is selected from the group consisting of deodorants, antiperspirants, antiperspirant/deodorants, including sprays, sticks and roll-on products, shaving products, skin lotions, moisturizers, toners, bath products, cleansing products, shampoos, conditioners, combined shampoo/conditioners, mousses, styling gels, hair sprays, hair dyes, hair color products, hair bleaches, waving products, hair straighteners, nail polish, nail polish remover, nail creams and lotions, cuticle softeners, sunscreen, insect repellent, anti-aging products, lipsticks, foundations, face powders, eye liners, eye shadows, blushes, makeup, mascaras, moisturizing preparations, foundations, body and hand preparations, skin care preparations, face and neck preparations, tonics, dressings, hair grooming aids, aerosol fixatives, fragrance preparations, aftershaves, make-up preparations, soft focus applications, night and day skin care preparations, non-coloring hair preparations, tanning preparations, synthetic and non-synthetic soap bars, hand liquids, nose strips, non-woven applications for personal care, baby lotions, baby baths and shampoos, baby conditioners, shaving preparations, cucumber slices, skin pads, make-up removers, facial cleansing products, cold creams, sunscreen products, mousses, spritzes, paste masks and muds, face masks, colognes and toilet waters, hair cuticle coats, shower gels, face and body washes, personal care rinse-off products, gels, foam baths, scrubbing cleansers, astringents, nail conditioners, eye shadow sticks, powders for face or eye, lip balms, lip glosses, hair care pump sprays and other non-aerosol sprays, hair-frizz-control gels, hair leave-in conditioners, hair pomades, hair de-tangling products, hair fixatives, hair bleach products, skin lotions, pre-shaves and pre-electric shaves, anhydrous creams and lotions, oil/water, water/oil, multiple and macro and micro emulsions, water-resistant creams and lotions, anti-acne preparations, mouth-washes, massage oils, toothpastes, clear gels and sticks, ointment bases, topical wound-healing products, aerosol talcs, barrier sprays, vitamin and anti-aging preparations, herbal-extract preparations, bath salts, bath and body milks, hair styling aids, hair-, eye-, nail-and skin-soft solid applications, controlled-release personal care products, hair conditioning mists, skin care moisturizing mists, skin wipes, pore skin wipes, pore cleaners, blemish reducers, skin exfoliators, skin desquamation enhancers, skin towelettes and cloths, depilatory preparations, personal care lubricants, nail coloring preparations, sunscreens, cosmetics, hair care products, skin care products, toothpastes, drug delivery systems for topical application of medicinal compositions that are to be applied to the skin and combinations comprising at least one of the foregoing applications.

13. The personal care formulation of claim 12 further comprising personal care ingredient selected from the group consisting of emollient, moisturizer, humectant, pigment, coated mica, colorant, fragrance, biocide, preservative, antioxidant, anti-microbial agent, anti-fungal agent, antiperspirant agent, exfoliant, hormone, enzyme, medicinal compound, vitamin, salt, electrolyte, alcohol, polyol, absorbing agent for ultraviolet radiation, botanical extract, surfactant, silicone oil, organic oil, wax, film former, thickening agent, particulate filler, clay, and combinations thereof.

14. The composition of claim 1 further comprising at least one compound selected from the group consisting of surfactants, emulsifiers, solvents, emollients, moisturizers, humectants, pigments, colorants, fragrances, biocides, preservatives, chelating agents, antioxidants, anti-microbial agents, anti-fungal agents, antiperspirant agents, exfoliants, hormones, enzymes, medicinal compounds, vitamins, alpha-hydroxy acids, beta-hydroxy acids, retinols, niacinamide, skin lightening agents, salts, electrolytes, alcohols, polyols, absorbing agents for ultraviolet radiation, botanical extracts, organic oils, waxes, film formers, thickening agents, particulate fillers, silicones, clays, plasticizers, humectants, occlusives, sensory enhancers, esters, other resins and film formers or film forming emulsifiers or high refractive index materials.

15. A process for producing a polysiloxane-based composition comprising:
i) mixing:
a) at least one polysiloxane of the general formula 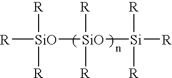
wherein each R is the same or different straight or branched open chain alkyl group containing from 1 to 12 carbon atoms, n is 0 to 5;
b) at least one carbomer; and,
c) water or an aqueous solution comprising at least 1 weight percent of the total weight of (a), (b) and (c).

16. The process of claim 15 wherein polysiloxane (a) is a polysiloxane having the formula: 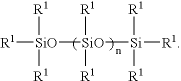
wherein R¹ is the same or different alkyl group containing from 6 to 10 carbon atoms, and n is 0 to 5.

17. The process of claim 15 wherein polysiloxane (a) is a polysiloxane having the formula:

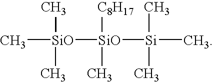

18. The process of claim 15 wherein polysiloxane (a) is a polysiloxane having the formula:

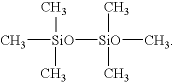

19. The process of claim 15 wherein polysiloxane (a) is present in an amount that ranges 0.01 to 99 weight percent of the total composition, carbomer (b) is present in an amount that ranges from 0.0001 to 40 weight percent of the total composition, and water or aqueous solution 1 to 99.5 weight percent of the total composition.

20. The process of claim 15 wherein polysiloxane (a) is present in an amount that ranges 0.1 to 15 weight percent of the total composition, carbomer (b) is present in an amount that ranges from 0.001 to 10 weight percent of the total composition, and water or aqueous solution 20 to 99 weight percent of the total composition.

21. The process of claim 15 wherein polysiloxane (a) is present in an amount that ranges 1 to 5 weight percent of the total composition, carbomer (b) is present in an amount that ranges from 0.02 to 5 weight percent of the total composition, and water or aqueous solution 60 to 98 weight percent of the total composition.

22. The process of claim 15 wherein polysiloxane (a) is at least one selected from the group consisting of 1,1,1,3,5,5,5-heptamethyl-3-ethyltrisiloxane; 1,1,1,3,5,5,5-heptamethyl-3-octyltrisiloxane; 1,1,1,3,5,5,5-heptamethyl-3-hexyltrisiloxane, 3-pentyl-1,1,1,3,5,5,5-heptamethyltrisiloxane; 1,1,1,3,5,5,5-heptamethyl-3-octyltrisiloxane, 1,1,1,3,5,5,5-heptamethyl-3-hexyltrisiloxane; 3-(1-ethylbutyl)-1,1,1,3,5,5,5-heptamethyltrisiloxane; 1,1,1,3,5,5,5-heptamethyl-3-(1-methylpentyl)trisiloxane; 1,1,1,3,5,5,5-heptamethyl-3-(1-methylpropyl)trisiloxane; 3-(1,1-dimethylethyl)-1,1,1,3,5,5,5-heptamethyltrisiloxane; 1,1,1,5,5,5-hexamethyl-3,3-bis(1-methylethyl)trisiloxane; 3-(3,3-dimethylbutyl)-1,1,1,3,5,5,5-heptamethyltrisiloxane; 1,1,1,3,5,5,5-heptamethyl-3-(3-methylbutyl)trisiloxane; 1,1,1,3,5,5,5-heptamethyl-3-(3-methylpentyl)trisiloxane; 1,1,1,3,5,5,5-heptamethyl-3-(2-methylpropyl)trisiloxane; 1,1,1,3,5,5,5-heptamethyl-3-propyltrisiloxane; 3-isohexyl-1,1,1,3,5,5,5-heptamethyltrisiloxane; 3-tert-pentyl-1,1,1,3,5,5,5-heptamethyltrisiloxane; 3-neo-pentyl-1,1,1,3,5,5,5-heptamethyltrisiloxane; 1,1,1,5,5,5-hexamethyl-3,3-dipropyltrisiloxane; 3,3-diethyl-1,1,1,5,5,5-hexamethyltrisiloxane; 3,3-dibutyl-1,1,1,5,5,5-hexamethyltrisiloxane; 3-ethyl-1,1,1,3,5,5,5-heptamethyltrisiloxane; 3-heptyl-1,1,1,3,5,5,5-heptamethyltrisiloxane; 1,1,1,5,5,5-hexamethyl-3,3-diethyltrisiloxane; 1,1,1,3,5,5,5-heptamethyl-3-butyltrisiloxane; and PEG/PPG 5/3 Methicone.

23. The process of claim 15 wherein carbomer (b) is at least one synthetic high molecular weight polymer of acrylic acid cross linked with either allyl esters of sucrose, pentaerythritol, or propylene.

24. The process of claim 15 wherein carbomer (b) is at least one synthetic high molecular weight polymer of acrylic acid cross linked with either alkyl esters of sucrose or pentaerythritol.

25. The process of claim 15 wherein carbomer (b) is a non-carbomer structurant or thickener.

26. The process of claim 25 wherein non-carbomer (b) is at least one selected from the group consisting of gums, cellulose derivatives, starches, and clays.

27. A personal care formulation comprising the polysiloxane-based composition prepared from the process of claim 15 where the personal care formulation is selected from the group consisting of deodorants, antiperspirants, antiperspirant/deodorants, including sprays, sticks and roll-on products, shaving products, skin lotions, moisturizers, toners, bath products, cleansing products, shampoos, conditioners, combined shampoo/conditioners, mousses, styling gels, hair sprays, hair dyes, hair color products, hair bleaches, waving products, hair straighteners, nail polish, nail polish remover, nail creams and lotions, cuticle softeners, sunscreen, insect repellent, anti-aging products, lipsticks, foundations, face powders, eye liners, eye shadows, blushes, makeup, mascaras, moisturizing preparations, foundations, body and hand preparations, skin care preparations, face and neck preparations, tonics, dressings, hair grooming aids, aerosol fixatives, fragrance preparations, aftershaves, make-up preparations, soft focus applications, night and day skin care preparations, non-coloring hair preparations, tanning preparations, synthetic and non-synthetic soap bars, hand liquids, nose strips, non-woven applications for personal care, baby lotions, baby baths and shampoos, baby conditioners, shaving preparations, cucumber slices, skin pads, make-up removers, facial cleansing products, cold creams, sunscreen products, mousses, spritzes, paste masks and muds, face masks, colognes and toilet waters, hair cuticle coats, shower gels, face and body washes, personal care rinse-off products, gels, foam baths, scrubbing cleansers, astringents, nail conditioners, eye shadow sticks, powders for face or eye, lip balms, lip glosses, hair care pump sprays and other non-aerosol sprays, hair-frizz-control gels, hair leave-in conditioners, hair pomades, hair de-tangling products, hair fixatives, hair bleach products, skin lotions, pre-shaves and pre-electric shaves, anhydrous creams and lotions, oil/water, water/oil, multiple and macro and micro emulsions, water-resistant creams and lotions, anti-acne preparations, mouth-washes, massage oils, toothpastes, clear gels and sticks, ointment bases, topical wound-healing products, aerosol talcs, barrier sprays, vitamin and anti-aging preparations, herbal-extract preparations, bath salts, bath and body milks, hair styling aids, hair-, eye-, nail-and skin-soft solid applications, controlled-release personal care products, hair conditioning mists, skin care moisturizing mists, skin wipes, pore skin wipes, pore cleaners, blemish reducers, skin exfoliators, skin desquamation enhancers, skin towelettes and cloths, depilatory preparations, personal care lubricants, nail coloring preparations, sunscreens, cosmetics, hair care products, skin care products, toothpastes, drug delivery systems for topical application of medicinal compositions that are to be applied to the skin and combinations comprising at least one of the foregoing applications.

28. The personal care formulation of claim 27 further comprising personal care ingredient selected from the group consisting of emollient, moisturizer, humectant, pigment, coated mica, colorant, fragrance, biocide, preservative, antioxidant, anti-microbial agent, anti-fungal agent, antiperspirant agent, exfoliant, hormone, enzyme, medicinal compound, vitamin, salt, electrolyte, alcohol, polyol, absorbing agent for ultraviolet radiation, botanical extract, surfactant, silicone oil, organic oil, wax, film former, thickening agent, particulate filler, clay, and combinations thereof.

29. The process of claim 15 further comprising at least one compound selected from the group consisting of surfactants, emulsifiers, solvents, emollients, moisturizers, humectants, pigments, colorants, fragrances, biocides, preservatives, chelating agents, antioxidants, anti-microbial agents, anti-fungal agents, antiperspirant agents, exfoliants, hormones, enzymes, medicinal compounds, vitamins, alpha-hydroxy acids, beta-hydroxy acids, retinols, niacinamide, skin lightening agents, salts, electrolytes, alcohols, polyols, absorbing agents for ultraviolet radiation, botanical extracts, organic oils, waxes, film formers, thickening agents, particulate fillers, silicones, clays, plasticizers, humectants, occlusives, sensory enhancers, esters, other resins and film formers or film forming emulsifiers or high refractive index materials.
